(19) United States
(12) Patent Application Publication
(10) Pub. No.: US 2018/0078199 A1
(43) Pub. Date: Mar. 22, 2018

(54) DETECTION OF SLEEP DISORDERED BREATHING USING CARDIAC AUTONOMIC RESPONSES

(71) Applicant: WAYNE STATE UNIVERSITY, Detroit, MI (US)

(72) Inventors: Abdulghani SANKARI, Bloomfield Hills, MI (US); M. Safwan BADR, West Bloomfield, MI (US); Scott MARESH, Brighton, MI (US)

(21) Appl. No.: 15/706,097

(22) Filed: Sep. 15, 2017

Related U.S. Application Data
(60) Provisional application No. 62/395,634, filed on Sep. 16, 2016.

(51) Int. Cl. A61B 5/00; A61B 5/0456; A61B 5/0205; A61B 5/1455; A61B 5/04; A61B 5/0432

(52) U.S. Cl. CPC A61B 5/4818; A61B 5/0456; A61B 5/0205; A61B 5/0255; A61B 5/04012; A61B 5/0432; A61B 5/14551

(57) ABSTRACT

A memory stores R-R interval (RRI) data collected from a patient over a time interval and oxygen saturation (SaO2) data collected from the patient over the time interval. A processor is programmed to analyze the SaO2 data to identify desaturation events, analyze the RRI data to identify dips, utilize the dips to construct a RRI dip index measure of RRI dips per unit time over the time interval, determine a number of desaturations above a predefined threshold, determine an oxygen desaturation index (ODI), and utilize the RRI dip index and the ODI to provide results indicative of a risk of sleep-disordered breathing (SDB) for the patient.

### CROSS-REFERENCE TO RELATED APPLICATIONS

This application claims the benefit of U.S. provisional application Ser. No. 62/395,634 filed Sep. 16, 2016, the disclosure of which is hereby incorporated in its entirety by reference herein.

### STATEMENT REGARDING FEDERALLY SPONSORED RESEARCH OR DEVELOPMENT

This invention was made with government support under Contract Nos. VA 11K2CX000547 and I01CX001040 awarded by the United States Department of Veterans Affairs. The Government has certain rights to the invention.

### TECHNICAL FIELD

Aspects of the disclosure generally relate to detection of sleep-disordered breathing using cardiac-autonomic responses.

### BACKGROUND

Sleep-disordered breathing (SDB) is associated with significant adverse health consequences such as hypertension (HTN) and cardiovascular disease (CVD). These consequences may, in some cases, be prevented with nasal continuous positive airway pressure (CPAP) therapy.

Sleep-disordered breathing (SDB) is characterized by the occurrence of recurrent episodes of apnea and hypopnea, resulting in oxyhemoglobin desaturation and sleep fragmentation. Apneic episodes are identified by the absence of flow, independent of immediate physiologic consequences. In contrast, the qualitative nature of clinical polysomnography precludes identification of hypopnea based on ventilatory parameters alone. The original definition of hypopnea in Gould's study included flow reduction associated with an oxyhemoglobin desaturation. Thus, all definitions of hypopnea are based on physiologic consequences of decreased alveolar ventilation, namely decreased flow, oxyhemoglobin desaturation, and transient cortical arousal.

Criteria for airflow reduction have also been variable across different studies, ranging from perceptible qualitative decrease in oral-nasal flow to 50% drop from baseline. Scoring of hypopnea according to the most recent American Academy of Sleep Medicine (AASM) scoring manual requires the presence of physiologic consequences, namely 3% desaturation and/or or cortical arousal. A common limitation of all definitions of hypopnea is that the magnitude of oxyhemoglobin desaturation depends on individual “host factors” such as body weight and baseline pulmonary function. Similarly, the presence of carboxyhemoglobin may shift the oxyhemoglobin dissociation curve to the left and hence dampen the magnitude of oxyhemoglobin desaturation in current smokers.

The Sleep Heart Health Study investigated the effect of varying the level of hypopnea-related oxyhemoglobin desaturation on the association between SDB and prevalent cardiovascular disease. Specifically, the frequency of hypopneas defined by a threshold of oxyhemoglobin desaturation of 4% or more was associated with cardiovascular disease, while lesser degree of desaturation or cortical arousal were not associated with cardiovascular disease based on self-report. However, there are other studies that have examined the immediate cardiovascular response to episodes of decreased flow, independent of the magnitude of desaturation or electroencephalogram (EEG) cortical arousal, and found conflicting results. While Ayappa et al. found less effect on heart rate (HR) increase immediately after non-apneic and mild reduction in flow, found that non-apneic events were associated more consistently with increased HR.

### SUMMARY

A new method of detecting heart accelerations during sleep that can improve the accuracy of diagnosing SDB without the need for EEG or a desaturation threshold. The method may utilize an automatic detection of heart rate accelerations obtained during sleep study that can be translated into an executable program or a plug □ in for sleep scoring software and can be used in any sleep study across the world. As explained in detail below, the algorithm can predict long term prognosis and incidence of cardiovascular diseases (such as heart attack, heart failure, or need for the cardiac procedures) and cardiovascular-related mortality.

In one or more illustrative embodiments, a system identifies risk of sleep-disordered breathing (SDB) from heart rate data without using electroencephalogram data or a desaturation threshold. In the system, a memory stores R-R interval (RRI) data collected from a patient over a time interval and oxygen saturation (SaO2) data collected from the patient over the time interval. A processor is programmed to analyze the SaO2 data to identify desaturation events, analyze the RRI data to identify dips, utilize the dips to construct a RRI dip index measure of RRI dips per unit time over the time interval, determine a number of desaturations above a predefined threshold, determine an oxygen desaturation index (ODI), and utilize the RRI dip index and the ODI to provide results indicative of a risk of sleep-disordered breathing (SDB) for the patient.

In one or more illustrative embodiments, a method for identifying a risk of sleep-disordered breathing (SDB) from heart rate data without using electroencephalogram data or a desaturation threshold includes receiving R-R interval (RRI) data collected from a patient over a time interval; dividing the RRI data into equal segments of a predefined time period length; dividing each data point of the RRI data by an average RRI value calculated for the segment in which the data point is included; if a ratio of the data point to the average RRI value is less than a predefined percentage, identifying the data point as being a dip; constructing a RRI dip index measure of RRI dips per unit time over the time interval; and utilizing the RRI dip index to provide results indicative of the risk of sleep-disordered breathing (SDB) for the patient.

In one or more illustrative embodiments, non-transitory computer-readable medium comprising instructions for identifying a risk of sleep-disordered breathing (SDB) from heart rate data without using electroencephalogram data or a desaturation threshold, that, when executed by a processor, cause the processor to receive R-R interval (RRI) data collected from a patient over a time interval; divide the RRI data into equal segments of a predefined time period length; divide each data point of the RRI data by an average RRI value calculated for the segment in which the data point is included; if a ratio of the data point to the average RRI value is less than a predefined percentage, identify the data point as being a dip; construct a RRI dip index measure of RRI dips per unit time over the time interval; and utilize the RRI dip index to provide results indicative of the risk of sleep-disordered breathing (SDB) for the patient.

### BRIEF DESCRIPTION OF THE DRAWINGS

FIG. 1 illustrates an example system for analyzing whether a patient has SDB;

FIG. 2 illustrates an example process for determining whether a patient has SDB according to analysis of SaO2 data and analysis of R-R interval (RRI) data;

FIG. 3 illustrates further details of the analysis of the SaO2 data to identify desaturation events;

FIG. 4 illustrates further details of the analysis of the RRI data to identify dips;

FIG. 5 illustrates an example graphical illustration of results of the process for determining whether a patient has SDB;

FIG. 6 illustrates an example RRI data response to a representative non-apneic event; and

FIG. 7 illustrates example data of adjusted time to event Cox proportional hazard models for RRDI predicting incidence of CVD events.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Sleep-disordered breathing (SDB) is strongly linked to cardiovascular morbidity and mortality. SDB traditionally include apneic and non-apneic respiratory events (RE) if associated with desaturation or arousals. However SDB encompasses a spectrum of respiratory abnormalities that could disrupt normal physiology.

Current technology in detecting and diagnosing sleep-related respiratory events is based either on full polysomnography, which requires in-laboratory testing, or on portable home sleep studies (HST). The HST method varies in level of complexity and data acquisition. Most HST combine pulse oxymetry and respiratory effort. However, a majority of respiratory events (e.g., hypopnea) can be missed if not followed by significant desaturation (e.g., equal to or more than 4%). According to the recommended scoring criteria of the American Academy of Sleep Medicine (AASM), hypopnea is defined as a drop of at least 30% of airflow associated with desaturation (equal to or more than 3%) and/or arousal. Respiratory events such apnea and hypopnea lead to sympathetic activation following each event that can lead to brief tachycardia as a result of arousal and/or desaturation.

As explained in detail below, using single lead electrocardiography (ECG) without electroencephalogram (EEG) monitoring, systems and methods may calculate an index correlated to severity of sleep disordered breathing. Using the index, severity of sleep disordered breathing may be determined to provide an estimate of the patient's long term outcome. For instance, the index may be used to identify patients with increased risk of adverse cardiovascular outcome, such as heart attack, heart failure, cardiac procedure, or cardiac death.

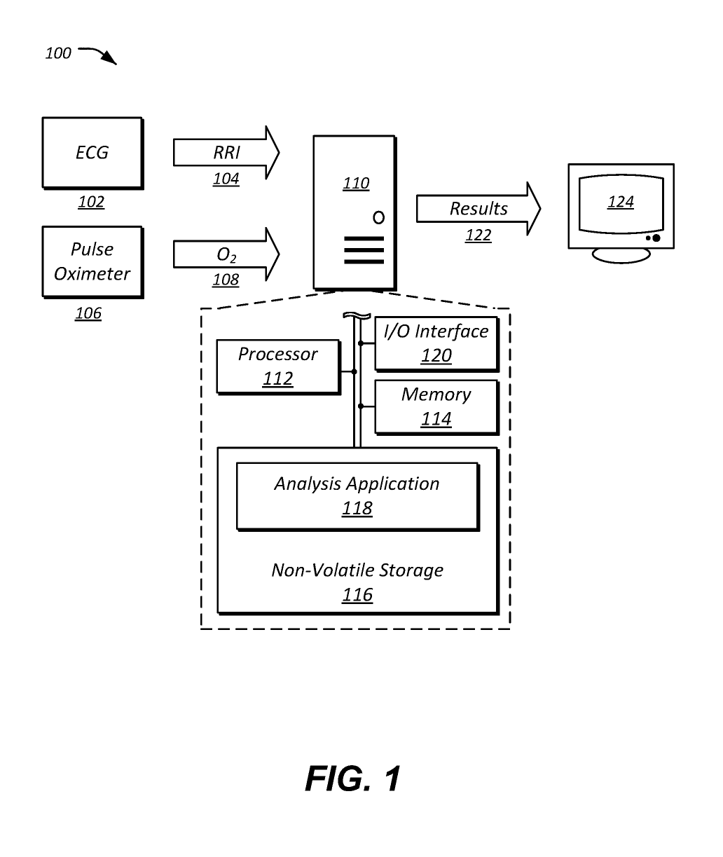

FIG. 1 illustrates an example system 100 for analyzing whether a patient has SDB.

The system includes an electrocardiography (ECG) device 102 configured to create RRI data 104, and a pulse oximeter 106 configured to create oxygen saturation (SaO2) data 108. The system 100 further includes a computing device 110 having a processor 112, a memory 114, a non-volatile storage 116, and an input/output interface 120. The analysis application 118 may be an application included on the storage 116 of the computing device 110. The computing device 110 receives the RRI data 104 and the oxygen saturation data 108 (e.g., via a wired or wireless transceiver), and executes the analysis application 118 to create results 122. The results 122 may be provided to a display 124 for review. It should be noted that the system 100 is merely one example, and systems including more, fewer, and/or different components may be used. For example, while the analysis application 118 is illustrated as a component of a standalone computer, in other examples the analysis application 118 may be implemented in software, firmware, and/or hardware in a medical device, and/or integrated into a non-diagnostic consumer device such as an application executed by a user's smartphone.

Cardiac autonomic disturbances during sleep, such as R-R interval (RRI), can serve as a predictor of CVD in patients with SDB. As explained below, increased RRI dip index (RRDI) during sleep is associated with increased CVD or death related to CVD. Accordingly, the analysis application 118 includes instructions that, when loaded into the memory 114 and executed by the processor 112, cause the computing device 110 to utilize the RRI data 104 and the SaO2 data to determine results 122 indicative of whether or not a patient has SDB. Specific examples of these instructions are described in detail with reference to the processes below.

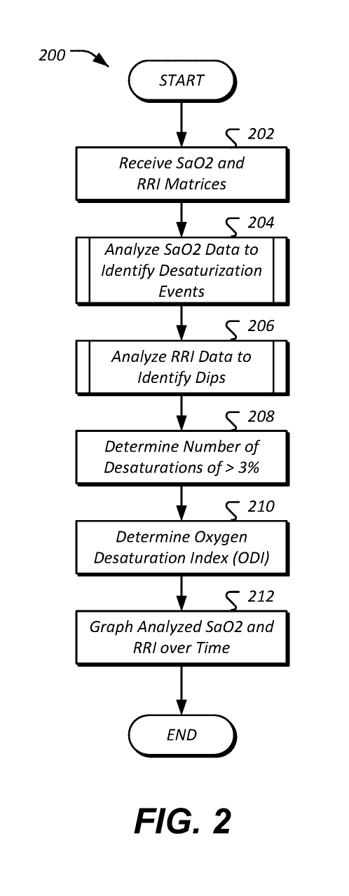

FIG. 2 illustrates an example process 200 for determining whether a patient has SDB according to analysis of SaO2 data 108 and analysis of RRI data 104. As discussed in detail, the analysis application 118 detects sleep disordered breathing using ECG tracing and beat-to-beat heart rate variability. Respiratory events are typically followed by a drop in RRI and a desaturation. Accordingly, the analysis application 118 analyzes changes in R-R interval (RRI) length (heart rate) and desaturations during sleep.

At 202, the analysis application 118 receives the SaO2 data 108 and RRI data 104. In an example, the data may be received from the ECG 102 and pulse oximeter 106 from a connected patient. In other examples, the SaO2 data 108 and RRI data 104 may be received from storage, e.g., having been collected from a previous recording of patient ECG tracing and beat-to-beat heart rate variability. In some examples, the analysis application 118 may perform preprocessing of the RRI data 104. This preprocessing may be done to clean the data for further processing. As some examples, the preprocessing may include normalizing heights of the raw RRI values, removing short artifacts, and adding missing beats, if necessary. In other examples, the analysis application 118 may receive RRI data 104 in which the raw RRI values have been manually reviewed for consistency. In some examples, one aspect of cleaning of the RRI data 104 includes removing indexed RRI data points from the RRI data 104. This removal may be utilized later in determining the total amount of RRI data 104 that was processed.

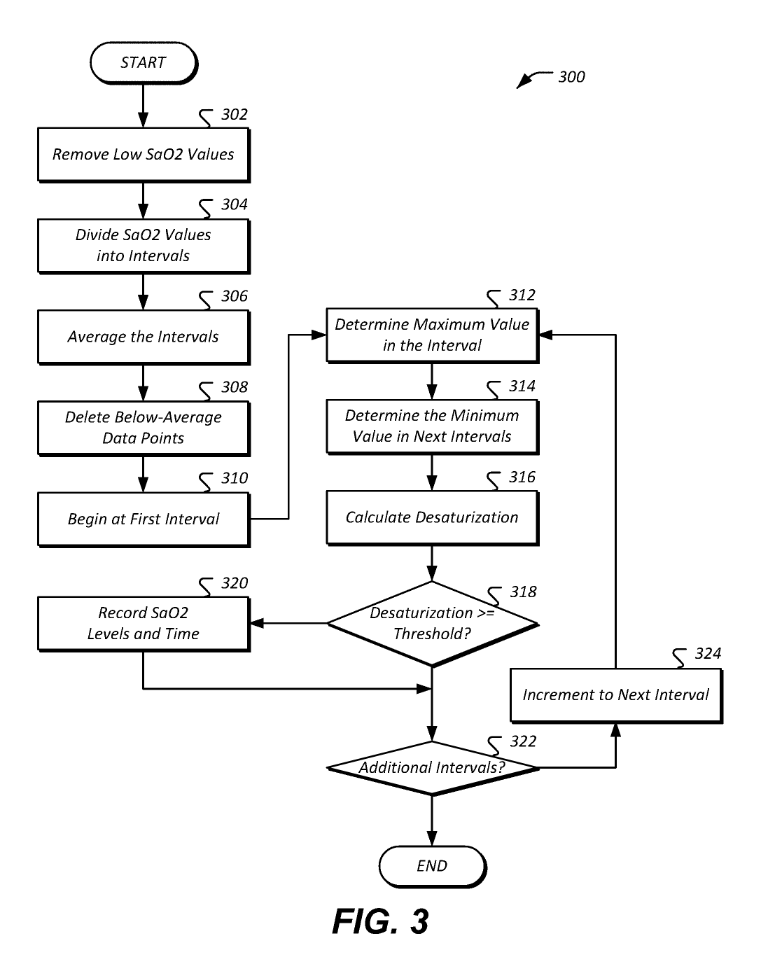

At 204, the analysis application 118 analyzes the SaO2 data 108 to identify desaturation events. Further aspects of the analysis are illustrated with reference to the process 300 of FIG. 3. Referring to FIG. 3, at 302 the analysis application 118 removes low SaO2 data 108 values. (e.g., those that are less than 50%). These values may be removed because these are likely to be artifacts, as values this low are rare or indicative of other health-related issues. In other examples, the threshold percentage for removal could be even higher, e.g., 60%, 70%, or even 80%. At 304, the analysis application 118 divides the SaO2 data 108 values into intervals (e.g., one second intervals). At 306, the analysis application 118 determines an average value for each of the intervals. At 308, the analysis application 118 deletes data points from the SaO2 data 108 that are more than a predefined amount below the average (e.g., that are 0.1% smaller than the average in an example). These removals may also be done to remove data artifacts, although this removal may not necessarily be directly tied to a specific physiological effect.

The analysis application 118 then cycles through the intervals. At 310, the analysis application 118 begins at the first interval. At 312, the analysis application 118 determines the maximum value in the next set of intervals (e.g., the minimum value across the next 60 intervals in an example). For instance, the analysis application 118 may review through the next samples of data until the time of the reviewed data is at least 60 intervals in length greater than the timestamp of the current maximum value (or the maximum amount available if less data than 60 intervals remains) to find a minimum. It should be noted that the use of a timeframe of 60 intervals is only one example, and other values, such as 30 or 90 times the interval length may be used. The analysis application 118 also calculates the desaturation for the interval at 316. For example, the analysis application 118 may subtract the maximum value of the interval from the minimum value identified for the set of intervals. If at 316 the analysis application 118 determines that the desaturation is at least a predefined value (e.g., 1%), the analysis application 118 proceeds to 320 to record or mark the SaO2 levels and time information for later processing. After 318 or 320, the analysis application 118 proceeds to 322 to determine whether additional intervals remain. If so, the analysis application 118 transitions to 324 to increment to the next interval, and to operation 312 to continue cycling through the intervals. If no further intervals remain, the process 300 ends and flow returns to the process 200.

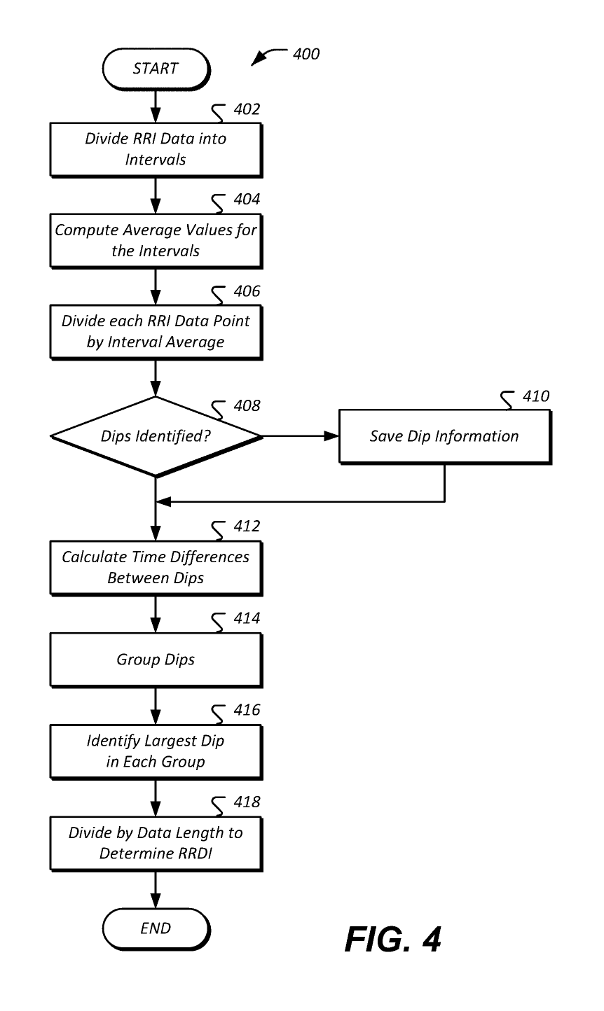

Referring back to FIG. 2, at 206 the analysis application 118 analyzes the RRI data 104 to identify “dips.” As explained in greater detail with reference to FIG. 4, at 402 the analysis application 118 divides the RRI data 104 into segments (e.g., one minute segments) and at 404 the analysis application 118 calculates the average RRI value for each segment. The analysis application 118 at 406 analyzes each point of the RRI data 104 and divides it by the average RRI value calculated for the segment in which the RRI data 104 point belongs. If the analysis application 118 determines at 408 that this ratio is less than a predefined threshold value, (e.g., 90%), at 410 the analysis application 118 stores or marks the interval index, RRI length, time at which the RRI length was found, and the ratio for further processing. It should be noted that 90% is merely one example, and other thresholds may additionally or alternately be determined, for example, 85%, 80%, 75%, 70%, 65% or 60% as some other possibilities. In cases where additional thresholds are used, the analysis application 118 may provide indices for each. Regardless, RRI values for which the ratio is less than the predefined threshold value may be referred to as “dips.”

At 412, the analysis application 118 calculates time differences between the dips. The analysis application 118 analyzes these dips in chronological order and places them into groups at 414. The analysis application 118 may optionally perform filtering of the dips to remove standalone dips. Standalone dips may refer to dips flanked on both sides by an RRI ratio above the predefined threshold (e.g., above the 90%). Such dips may be removed as they do not contribute to a greater trend of decreasing or increasing RRI ratio. Regardless of whether the filtering is performed, the analysis application 118 creates a group and places all dips in it until the analysis application 118 identifies a pair of dips separated by more than a predefined time interval (e.g., ten seconds in an example, although greater or lesser time periods may be used). If so, the analysis application 118 creates a new group for the separated dip. The analysis application 118 analyzes and groups all of the “dips” in this manner. Using the determined groups, at 416 the analysis application 118 identifies the largest dip (i.e., the one with the smallest ratio) in each group and notes these as the biggest dips per group.

At 418, the analysis application 118 divides the number of the biggest dips in the overall data file by the total time length of the file to determine the RRDI (RR Interval Dips Index). The RRDI may refer to a measure of the overall events per unit time, e.g., per hour in some examples. As mentioned above, one aspect of cleaning of the RRI data 104 may include removing indexed data points for artifact data from the RRI data 104. When determining the RRDI, the analysis application 118 may detect when intervals have been removed if there is a skip in the index column (for example, for the indices [1, 2, 3, 5, 6], index 4 was skipped). The analysis application 118 may, accordingly, determine an amount of time elapsed between the pairs of data points flanking each skipped data point, sums these amounts of time, and subtracts the sum from the total length of the study. This difference may be referred to as the “corrected time.” The analysis application 118 may, accordingly, use the corrected time as the total time length in determining the overall RRDI. After 418, the process 400 ends and flow returns to the process 200.

Generally, an increased RRDI measure may correlate to an increased risk of adverse cardiovascular outcome, such as heart attack, heart failure, cardiac procedure, or cardiac death. Accordingly, the RRDI measure may be compared against a cutoff value to determine whether the patent whose data is being analyzed should be flagged for follow-up screening. This cutoff value may be scaled according to various factors, such as patient age, gender, weight, or other demographic or health risk information (e.g., smoker vs. non-smoker).

Referring back to FIG. 2, at 208 the analysis application 118 determines a number of desaturations that are greater than or equal to a predefined amount (e.g., greater than 3%). At 210, the analysis application 118 calculates the oxygen desaturation index (ODI), which refers to a measure of the number of events identified at 208 over time. For instance, the ODI may refer to an hourly index of the number of 3% events per hour. At 212, using the RRI data 104, the SaO2 data 108, and the information marked or stored above, the analysis application 118 generates the results 122. These results 122 may be provided to the display 124.

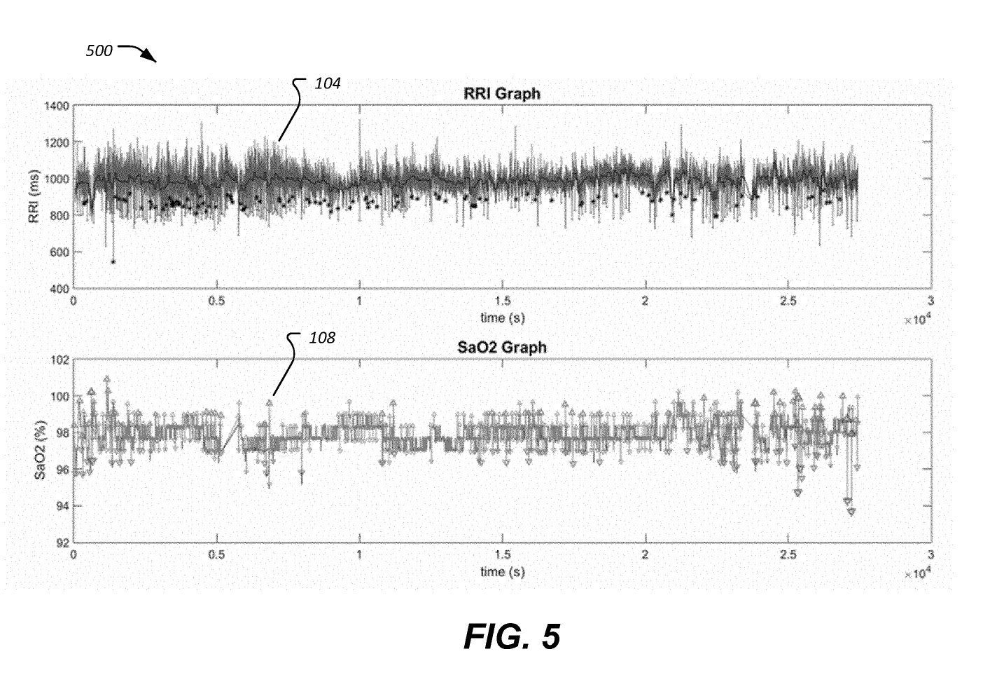

FIG. 5 illustrates an example graphical illustration 500 of the results 122 of the process 200 for determining whether a patient has SDB. As shown in the illustration 500, the RRI data 104 and the SaO2 data 108 are graphed over time with the same time scale.

More specifically, the RRI data 104 may be graphed to illustrate the raw data before processing (e.g., shown in gray). The RRI data 104 may also be illustrated with the calculated average (e.g., a minute average computed at operation 306). This average is shown in black. The dips computed at operation 410 may also be shown, indicated in the illustration as the dark dots for dips to be considered and as the black asterisks for standalone dips that are filtered out.

Regarding the SaO2 data 108, the raw data may be displayed along with the relative maximums and minimums. For instance, indications of a first size and/or color (e.g., large and red triangles) may indicate maximums or minimums of 3% or more, indications of a second size and/or color (e.g., medium and purple triangles) may indicate maximums or minimums of 2% to 3%, and indications of a third size and/or color (e.g., small and blue triangles) may indicate maximums or minimums of 1% to 2%. Continuing with the example of triangular indications, upward pointing triangles may refer to relative maximums, while downward pointing may triangles refer to relative minimums.

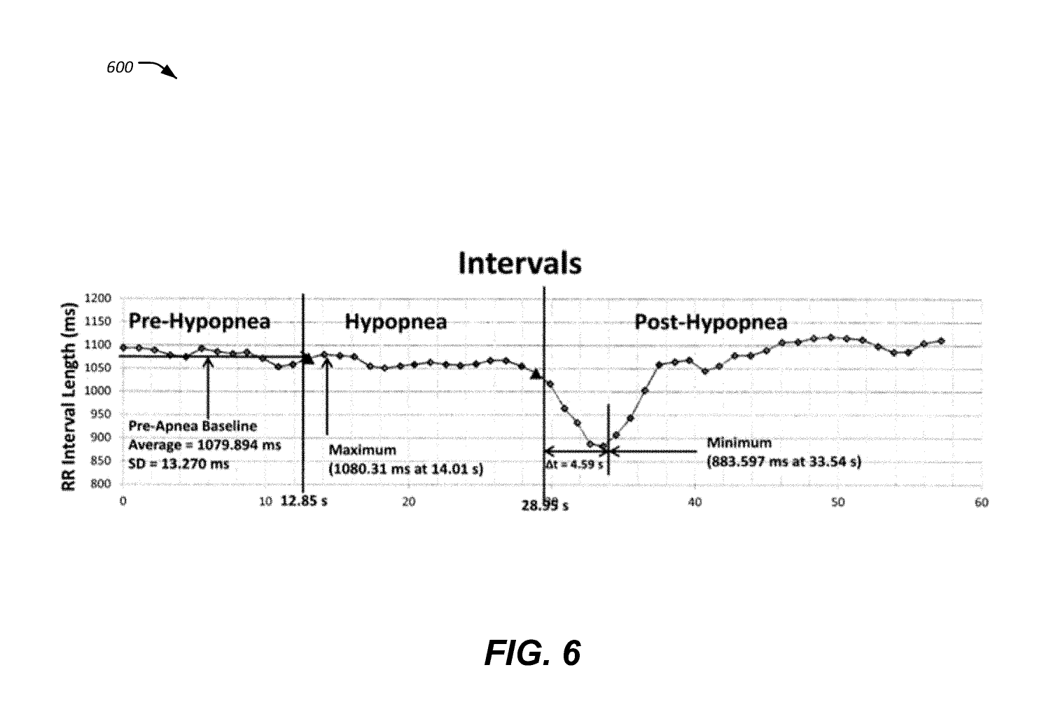

FIG. 6 illustrates an example RRI response to a representative non-apneic event. Using the analysis application 118, the system 100 may more accurately detect respiratory events without EEG monitoring. This detection may be done based on the magnitude of drop in RRI following respiratory event >/=10 from a preceding baseline. Notably, this determination may be made independent of desaturation or arousal on EEG. Accordingly, the system 100 may further allow for automated scoring of sleep studies that were recorded in-laboratory or at home. By simple run of the analysis application 118 using single lead ECG, the system 100 may calculate the RRDI index to estimate severity of sleep disordered breathing and thereby estimate the patient's long term outcome, such as CVD.

While many of the examples described herein relate to the analysis of ECG data, it should be noted that other sources of heart data may also be used. In an example, instead of RRI data 104, heart rate data or another type of heart beat information may potentially be used to identify the greatest dips per measure of percent of heart beats.

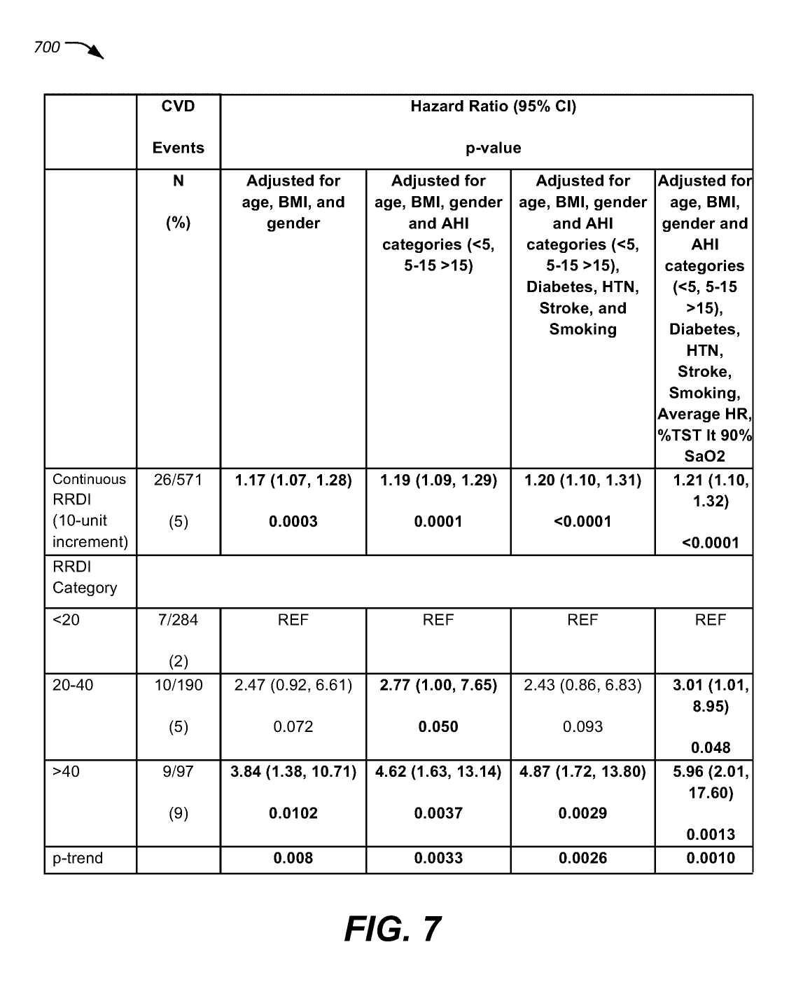

FIG. 7 illustrates example data 700 of adjusted time to event Cox proportional hazard models for RRDI predicting incidence of CVD events. CVD events may include, as some examples, heart attack, angioplasty, heart failure, stent, pacemaker, coronary artery disease, bypass surgery, atherosclerosis, or CVD death.

Regarding the data set, the proportional hazards regression is based on a sample size of 571 persons. Out of an initial set of 1,397 studies of persons, 141 were excluded as having used beta blockers during polysomnography (PSG), while 84 were excluded as having used CPAP on the night of the study. Thus, 1,172 scored PSG studies remained from 745 individuals. Out of these, 70 were excluded as having had no follow-up, while 46 were excluded as having had an event before the PSG. Thus, 629 individuals with PSG data remained. Out of these, 58 individuals were excluded as having used beta blockers at any other time during the study. Accordingly, a remaining sample size of 571 individuals with PSG data were included in the study.

As shown in the data 700, 26 out of the 571 individuals displayed continuous RDDI events and experienced a CVD event. Two hundred eighty-four of the 571 individuals had an RRDI index of less than 20, where out of those seven experienced a CVD event (2%). One hundred ninety of the 571 individuals had an RDDI index between 20 and 40, where out of those 10 experienced a CVD event. Ninety-seven of the 571 individuals had an RDDI index of greater than 40, where out of those nine experienced a CVD event (9%).

As can be seen by the hazard ratios, out of the 26 individuals who experienced a CVD event, the individuals showing continuous RDDI events were 1.17 times more likely to experience a CVD event. For those with an RDDI score of 20-40, the likelihood increases to 2.47 times, and for those with an RDDI score exceeding 40, the likelihood increases to 3.84 times. These likelihoods become even more significant once additional factors such as AHI categories, diabetes, HTN, stroke, smoking, average HR, and % TST is 90% SaO2 are accounted for. Accounting for these additional factors, individuals showing continuous RDDI events were 1.21 times more likely to experience a CVD event. For those with an RDDI score of 20-40, the likelihood increases to 3.01 times, and for those with an RDDI score exceeding 40, the likelihood increases to 5.96 times. Thus, the aforementioned systems and methods of detecting HR accelerations during sleep improve the accuracy of diagnosing SDB without the need for EEG or desaturation thresholds. Accordingly, the systems and methods allow for automated scoring of sleep studies that can be recorded in the laboratory or at home.

Computing devices described herein, such as the computing device 110, generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C#, C++, Visual Basic, Java Script, Perl, MatLab, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for identifying a risk of sleep-disordered breathing (SDB) from heart rate data without using electroencephalogram data or a desaturation threshold, comprising:
a memory storing R-R interval (RRI) data collected from a patient over a time interval and oxygen saturation (SaO2) data collected from the patient over the time interval; and
a processor programmed to
analyze the SaO2 data to identify desaturation events,
analyze the RRI data to identify dips,
utilize the dips to construct a RRI dip index measure of RRI dips per unit time over the time interval,
determine a number of desaturations above a predefined threshold,
determine an oxygen desaturation index (ODI), and
utilize the RRI dip index and the ODI to provide results indicative of the risk of sleep-disordered breathing (SDB) for the patient.

2. The system of claim 1, wherein the RRI data is received from an electrocardiography (ECG) device, and the SaO2 data is received from a pulse oximeter device.

3. The system of claim 1, wherein the processor is further programmed to:
divide the RRI data into equal segments of a predefined time period length;
divide each data point of the RRI data by an average RRI value calculated for the segment in which the data point is included; and
if a ratio of the data point to the average RRI value is less than a predefined percentage, identify the data point as being one of the dips.

4. The system of claim 3, wherein the processor is further programmed to, for each identified dip, identify an interval index of the dip, an RRI length of the dip, a time at which the RRI length was found, and the ratio of the data point to the average RRI value.

5. The system of claim 3, wherein the time period length is one minute.

6. The system of claim 3, wherein the predefined percentage is 90%.

7. The system of claim 3, wherein the processor is further programmed to:
chronologically place dips into a group until a pair of dips in the group are separated by more than a predefined group time interval;
responsive to the pair of dips being separated by more than a predefined time interval, create a new group for continuing the chronological placement of dips; and
for each group, identify a dip having the smallest ratio in each group; and
compute a respiratory-related RRI drops value as a count of the dips having the smallest ratio in each group.

8. The system of claim 7, wherein the predefined group time interval is ten times the time period length.

9. The system of claim 8, wherein the predefined group time interval is ten minutes.

10. A method for identifying a risk of sleep-disordered breathing (SDB) from heart rate data without using electroencephalogram data or a desaturation threshold, comprising:
receiving R-R interval (RRI) data collected from a patient over a time interval;
dividing the RRI data into equal segments of a predefined time period length;
dividing each data point of the RRI data by an average RRI value calculated for the segment in which the data point is included;
if a ratio of the data point to the average RRI value is less than a predefined percentage, identifying the data point as being a dip;
constructing a RRI dip index measure of RRI dips per unit time over the time interval; and
utilizing the RRI dip index to provide results indicative of the risk of sleep-disordered breathing (SDB) for the patient.

11. The method of claim 10, wherein the time period length is one minute.

12. The method of claim 10, wherein the predefined percentage is 90%.

13. The method of claim 10, further comprising:
chronologically placing dips into a group until a pair of dips in the group are separated by more than a predefined group time interval;
responsive to the pair of dips being separated by more than a predefined time interval, create a new group for continuing the chronological placement of dips; and
for each group, identify a dip having the smallest ratio in each group; and
compute a respiratory-related RRI drops value as a count of the dips having the smallest ratio in each group.

14. The method of claim 13, wherein the predefined group time interval is ten times the time period length.

15. A non-transitory computer-readable medium comprising instructions for identifying a risk of sleep-disordered breathing (SDB) from heart rate data without using electroencephalogram data or a desaturation threshold, that, when executed by a processor, cause the processor to:
receive R-R interval (RRI) data collected from a patient over a time interval;
divide the RRI data into equal segments of a predefined time period length;
divide each data point of the RRI data by an average RRI value calculated for the segment in which the data point is included;
if a ratio of the data point to the average RRI value is less than a predefined percentage, identify the data point as being a dip;
construct a RRI dip index measure of RRI dips per unit time over the time interval; and
utilize the RRI dip index to provide results indicative of the risk of sleep-disordered breathing (SDB) for the patient.

16. The medium of claim 15, wherein the time period length is one minute.

17. The medium of claim 15, wherein the predefined percentage is 90%.

18. The medium of claim 15, further comprising instructions that, when executed by the processor, cause the processor to:
chronologically place dips into a group until a pair of dips in the group are separated by more than a predefined group time interval;
responsive to the pair of dips being separated by more than a predefined time interval, create a new group for continuing the chronological placement of dips; and
for each group, identify a dip having the smallest ratio in each group; and
compute a respiratory-related RRI drops value as a count of the dips having the smallest ratio in each group.

19. The medium of claim 18, wherein the predefined group time interval is ten times the time period length.
